(19) United States
(12) Patent Application Publication
(10) Pub. No.: US 2003/0033011 A1
(43) Pub. Date: Feb. 13, 2003

(54) Intraocular lens for vision correction and cataract prevention

(76) Inventors: Jack A. Singer, Randolph, VT (US); Joe Kiernan, Randolph, VT (US)

Correspondence Address: JENNER & BLOCK, LLC, ONE IBM PLAZA, CHICAGO, IL, 60611, US

(21) Appl. No.: 09/924,563

(22) Filed: Aug. 8, 2001

(51) Int. Cl. A61F 2/16

(52) U.S. Cl. 623/6.6; 623/6.44; 623/6.4

(57) ABSTRACT

An intraocular lens (IOL) corrects vision disorders and prevents the formation of cataracts. The IOL can be inserted in the anterior or posterior chamber of the eye, or can be iris-fixated. The IOL can correct for myopia, hyperopia, presbyopia and/or astigmatism. Additionally, the IOL contains an ultraviolet radiation (UVR) blocker, that absorbs UVR in the 300-400 nm range. The absorption of the UVR allows the IOL to reduce or eliminate cataract formation.

### BACKGROUND OF THE INVENTION

1. Field of the Invention

This invention relates to intraocular lenses, and more particularly, to an intraocular refractive correction lens that corrects eyesight and contains an ultraviolet radiation (“UVR”) absorber that can reduce or eliminate cataract formation, and to a method of implanting an intraocular lens (“IOL”) to correct eyesight and reduce or eliminate cataract formation.

2. Description of the Related Art

The number one cause of blindness in the world is cataracts. A cataract is any change in the structure of the natural crystalline lens in the eye that leads to a loss of transparency. Although factors such as nutrition and genetics play a role in cataract formation, UVR exposure is primarily responsible. Ultraviolet light exposure has been proven to promote cataract formation. The clouding of the lens is irreversible, and once the cataracts begin to impair daily activities, the only treatment is surgical removal of the lens.

The formation of cataracts probably involves a number of physiological factors. However, a high correlation between cataract incidence and solar radiation, as well as the known cataract producing effects of oxygen, suggests that free radical exposure results in a cascade of toxic reactions leading to cataract formation.

Epidemiological and clinical evidence shows a link between UVR overexposure and cortical cataracts. The mechanism may be lens epithelial cell death rather than a disruption of equatorial cells, as had been thought. Early and complete protection from environmental UVR may help forestall the formation of cortical cataracts.

It is well accepted that the exposure to free radicals causes many of the changes in lens proteins throughout life. Proteins within the lens are damaged, probably by repeated exposure to ultraviolet light and oxygen. These modifications might lead to the formation of protein “clumps” that scatter light and contribute to the development of cataracts. The lens takes the brunt of this exposure since one of its prime functions is to serve as an optical filter to minimize the amount of UV light the retina receives. However, this filtering process also subjects the lens to constant exposure to free radicals and potential free radical damage.

The fraction of sunlight of most concern is the long wave, or near ultraviolet range, which is characterized by wavelength of 300-400 nanometers (nm). This band of ultraviolet radiation is known to cause damage to the eye by inducing chemical changes in the lens and retina. Though short wavelength light with wavelengths below 300 nm typically does not reach the earth's surface because of the atmospheric ozone layers, most of the long wave ultraviolet radiation in the 300-400 nm range is capable of penetrating to the surface of the earth.

Various parts of the eye absorb portions of the incident light that strikes the eyes so that only the unabsorbed or transmitted portions reach the retina. The cornea generally absorbs wavelengths up to about 340 nm. The natural crystalline lens absorbs most of the ultraviolet wavelengths between 300 and 400 nm. Other parts of the eye absorb portions of the visible spectrum. It is thought by the inventors that the natural lens can be protected, and cataracts delayed or prevented, if an artificial lens absorbs the UV radiation that would otherwise be absorbed by the natural lens.

For those persons who have had their natural lens removed, for example as a result of cataracts, injury or disease, a condition known as aphakia, UV light is no longer absorbed, but is instead transmitted to the retina. Lenses used to replace the natural lens such as IOLs usually contain compounds that function as UV absorbers, preventing the transmission of wavelengths of between 300-400 nm to the retina.

For sunglasses intended to provide ultraviolet protection, it is important that the lenses block a wide range of UV radiation, including UV radiation with wavelengths below 380 nm. In fact, recent concern on the effects of UV radiation to the eye has indicated a desire that sunglasses effectively block radiation with wavelengths lower than 400 nm.

Researchers involved in the study of lens implants in the phakic eye are focused on vision correction. The prior art discloses IOLs for placement in either the posterior or anterior chamber of the eye or for iris-fixated placement. However, most of these IOLs are made of polymethylmethacrylate (“PMMA”), a hard material. A hard material requires a larger incision for implantation, which causes more trauma to the eye. Almost all of the lenses in the prior art are merely for treatment of nearsightedness (myopia), and very rarely for farsightedness (hyperopia) or astigmatism.

There is no known lens in the art that can correct presbyopia or astigmatism. Moreover, there is no known lens that can treat myopia, hyperopia, compound myopic astigmatism, compound hyperopic astigmatism, or astigmatism in combination with presbyopia.

There is no known lens in the art that can prevent or delay the onset of cataract formation, or prevent or delay the onset of presbyopia.

Thus, it would be desirable to have an IOL that can treat the disorders of myopia, hyperopia, astigmatism and presbyopia, alone or in combination. Ideally, such a lens would be deformable, so that it could be implanted in a patient's eye with a smaller incision than that required by a hard lens. In addition, the lens should contain UV-absorbing materials, which can serve the multiple purposes of preventing cataract formation, delaying the onset of presbyopia, and preventing the degradation of the IOL.

### SUMMARY OF THE INVENTION

The method of the present invention can correct presbyopia and astigmatism, alone or in combination, by placing an IOL in the phakic eye of the patient. In addition to these conditions, the method of the present invention can correct the combination of astigmatism and myopia or astigmatism and hyperopia.

A lens in accordance with the present invention is made of a deformable material, such as silicone, hydrogel, collagen/acrylic blends, acrylic, or collagen/hema blends (collamer). Use of such materials causes less trauma to the eye during surgery because the lens must be inserted into the eye through an incision, and a such a lens can be deformed to fit into an incision smaller than the lens.

Moreover, by placing a UV-absorbing compound in the lens, the formation of cataracts and the onset of presbyopia can be delayed or prevented. These UV absorbing compounds may also extend the life of the IOL.

In one embodiment of the method of the present invention, an IOL that can improve vision, as well as absorb harmful UV radiation is placed in the posterior chamber of a phakic eye. This promotes the overall health of the eye by delaying cataract formation, presbyopia, and degradation of the IOL.

Additionally, a lens in accordance with the present invention can correct hyperopia, presbyopia and astigmatism through the use of an anterior chamber placed or iris-fixated IOL.

A lens in accordance with the present invention can correct hyperopia with a biconvex or convex-plano optic. Astigmatism is corrected with a toric element in the lens optic.

### DESCRIPTION OF THE DRAWINGS

FIG. 1 is a section view of an eye;

FIG. 2 is a plan view of an intraocular lens for placement in the posterior chamber of the eye in accordance with the present invention;

FIG. 3 is a section view of the intraocular lens of FIG. 2 placed in the posterior chamber of the eye;

FIG. 4 is a perspective view of another intraocular lens for placement in the posterior chamber of the eye in accordance with the present invention;

FIG. 5 is a section view of the intraocular lens of FIG. 4 placed in the posterior chamber of the eye;

FIG. 6 is a plan view of an intraocular lens for placement in the anterior chamber of the eye in accordance with the present invention;

FIG. 7 is a section view of the intraocular lens of FIG. 6 placed in the anterior chamber of the eye;

FIG. 8 is a plan view of an intraocular lens for iris fixated placement in the eye in accordance with the present invention;

FIG. 9 is a section view of the intraocular lens of FIG. 8 fixated on the iris;

FIG. 10 is a plan view of an intraocular lens optic for the treatment of presbyopia in accordance with the present invention; and

FIG. 11 is a plan view of an intraocular lens optic according to the invention showing the toric element for astigmatism correction.

### DETAILED DESCRIPTION OF THE PREFERRED EMBODIMENT

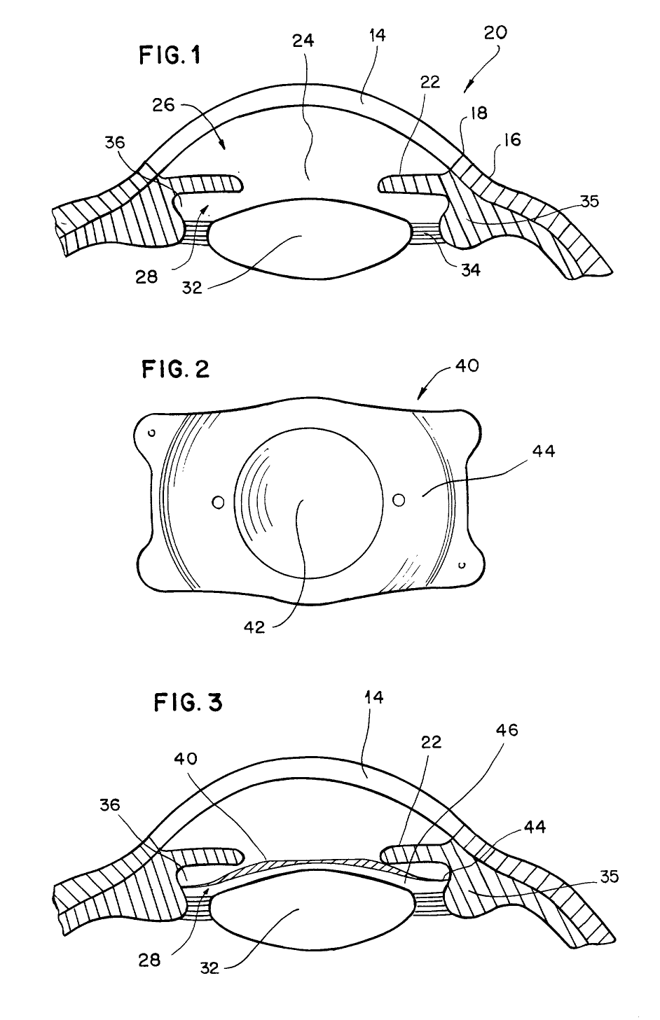

FIG. 1 illustrates the major components of the anterior segment of an eye 20. The major ocular components of an eye include a retina (not shown) and a cornea 14. Cornea 14 connects to a sclera 16 at a limbus 18. The anterior segment of eye 20 is divided into two principle chambers by an iris 22 and a pupil 24. Cornea 14 and iris 22 define an anterior chamber 26. Iris 22, a natural crystalline lens 32 and zonules 34 define a posterior chamber 28.

Natural crystalline lens 32 is located behind pupil 24 as defined by iris 22. Natural crystalline lens 32 is attached to a ciliary body 35 at its periphery by zonules 34. Eye 20 is deformable and zonules 34 allow lens 32 to deform to achieve a required focus to ensure that an image falls directly on the retina. Spectacles or contact lenses are required to compensate for errors in the focus of lens 32 during accommodation or axial length of the eye. Also present in the anterior segment of eye 20 is a ciliary sulcus 36.

As can be seen in FIGS. 2, 4, 6 and 8, lenses 40, 40′40″, and 40″′, respectively, in accordance with the present invention can come in several different shapes. Lens 40 of the present invention comprises two main elements: an optic element 42 and a haptic element 44. Optic element 42 consists of the portion of lens 40 which is in the field of vision of the wearer and thus provides vision correction. Optic element 42 also can include a UV radiation absorbing compound.

Haptic element 44 surrounds optic element 42 and does not participate in vision correction. Rather, haptic element 44 positions lens 40. Haptic element 44 can either be a single element, for example a circular element, or multiple elements, for example legs extending from lens 40. For lenses with one haptic, the maximum diagonal haptic dimension refers to the maximum measurable distance between two points on the haptic. For haptics with multiple elements, the maximum diagonal haptic dimension is the straight diagonal distance from the distal end of one haptic to the distal end of an opposite haptic. Haptic element 44 also can include a UV-absorbing compound, which would delay the onset and progression of degradation of the supporting elements of an IOL.

The present invention also advantageously uses UV-absorbing materials for corrective lenses 40 in the phakic eye. It is known that UV light can increase the chance of cataract formation. The present invention reduces the likelihood of cataract formation, a common problem for people who are exposed to UV radiation found in sunlight, by using UV absorbing materials in lens 40. It is known that the presence of an IOL in a phakic eye can increase the chance of cataract formation. The present invention also reduces the likelihood of IOL-induced cataract formation by reducing the additive effects of UV radiation and the presence of an IOL within a phakic eye.

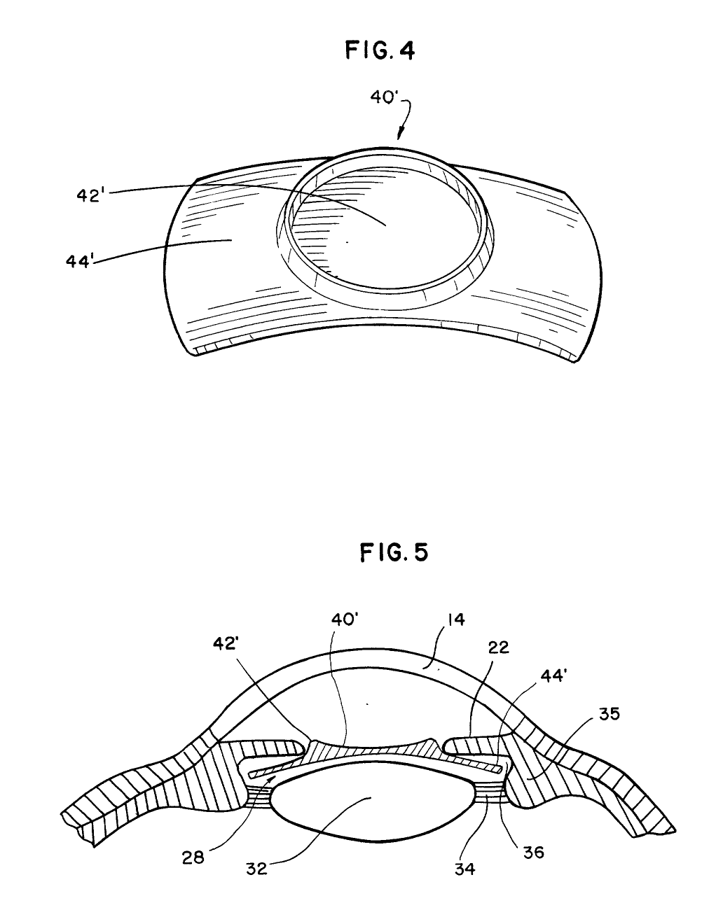

Turning now to FIGS. 2 and 4, alternate embodiments of the invention are shown in which lens 40 or 40′ is designed for placement in posterior chamber 28 of the anterior segment of an eye 20. Lens 40 or 40′ can be constructed of a material that is deformable. Such materials include silicone, hydrogel, collagen/acrylic blends, acrylic, and collagen/hema blends (collamer). Use of such material allows lens 40 or 40′ to be inserted through a smaller incision, which causes less trauma to the eye. Optic element 42 or 42′ of lens 40 or 40′ respectively can be constructed to be either a negative refracting lens or a positive refracting lens. Additionally, optic element 42 or 42′ can include a toric element (not shown) for astigmatism correction.

Lens 40 or 40′ for placement in posterior chamber 28 of anterior segment of an eye 20 can be held in place by either of two methods. As shown in FIG. 3, a first method calls for the diagonal haptic dimension of lens 40 to be greater than the diameter of ciliary sulcus 36, allowing haptic element 44 to contact ciliary sulcus 36. This contact is required for adequate vaulting of lens 40 in order to maintain a gap 46 between lens 40 and the anterior surface of crystalline lens 32. In this method, the maximum diagonal haptic dimension of haptic element 44 of lens 40 is determined by the size of the wearer's eye, and can be any size from under 10 to over 15 mm, but is preferably from 10.5 to about 14.0 mm.

A second method for securing lens 40′ for placement in posterior chamber 28 of anterior segment of an eye 20 is shown in FIG. 5. This method requires the diagonal haptic dimension of lens 40′ to be less than the diameter of ciliary sulcus 36, thus haptic element 44′ cannot contact ciliary sulcus 36. The interaction between optic element 42′ and pupil results in centration of lens 40′. The radius of curvature of lens 40′ creates a Bernoulli effect, which helps float lens 40′ anteriorly, away from the natural crystalline lens 32. In this method, haptic element 44′ can flare out in width in order to maximize the ability of lens 40′ to float freely in the eye, without interfering with the eye's natural crystalline lens 32. In this method, the maximum diagonal haptic dimension of haptic element 44′ of lens 40′ is again determined by the needs of the wearer, and can range from under 10 to over 12 mm, but is preferably in the range from about 10.0 to about 11.8 mm.

In addition to the above disclosed properties, lens 40 or 40′ can also contain a UV absorbing compound. See U.S. Pat. Nos. 5,133,745 and 4,528,311, incorporated herein by reference, for examples of effective UV absorbing compounds and methods for incorporating them into plastics. A compound that absorbs light in the 300-400 nm range is believed to be the most beneficial for reducing or preventing the formation of cataracts, delaying or preventing the onset of presbyopia, and preventing or delaying the degradation of the IOL.

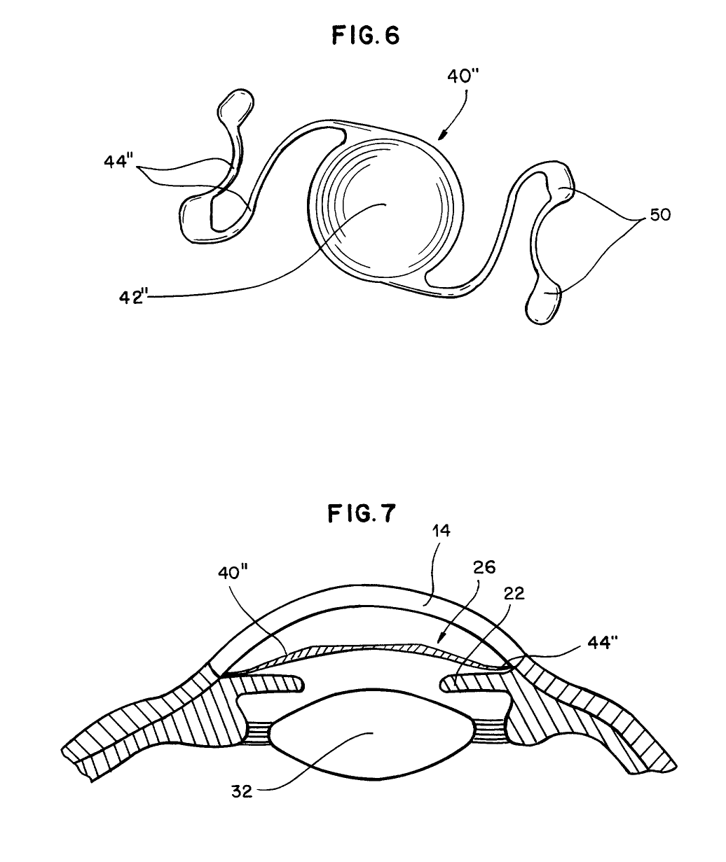

Referring now to FIGS. 6 and 7, a third embodiment of the invention is shown in which lens 40″ is designed for placement in anterior chamber 26 of anterior segment of an eye 20. Lens 40″ can be constructed of a material that is deformable, as previously described. Optic element 42″ of lens 40″ can be constructed to be either a negative refracting lens or a positive refracting lens. Additionally, optic element 42″ can include a toric element (not shown) for astigmatism correction.

Optic element 42″ of lens 40″ for placement in anterior chamber 26 of anterior segment of an eye 20 has a diameter determined by the needs of the wearer, and can range from about 5 to about 8 mm, but is preferably from 5.5 to 7.0 mm. Lens 40″ can have two haptic elements 44″, preferably made of flexible PMMA or acrylic. Each haptic element 44″ has two footplates 50, thereby providing a 4-point fixation in the anterior chamber angle. Haptic elements 44″ suspend lens 40″ in anterior chamber 26 of anterior segment of an eye 20 at a vault angle above 0 and up to several degrees, but preferably at a vault angle of about 2 to 5 degrees.

Implantation of lens 40″ in anterior chamber 26 of anterior segment of an eye 20 can be aided by the use of a flexible lens glide (not shown). The maximum diagonal haptic dimension of lens 40″ can vary from 10 mm to over 15 mm to permit proper fitting, but a range from 11.5 mm to 15.0 mm in 0.5 mm steps is sufficient to address most eyes. The length of lens 40″ is to be determined by adding 1.0 mm to the horizontal corneal diameter (white-to-white) measurement, in mm. No positioning holes are needed in optic element 42″ or haptic element 44″.

Lens 40″ also can contain a UV-absorbing compound as previously described.

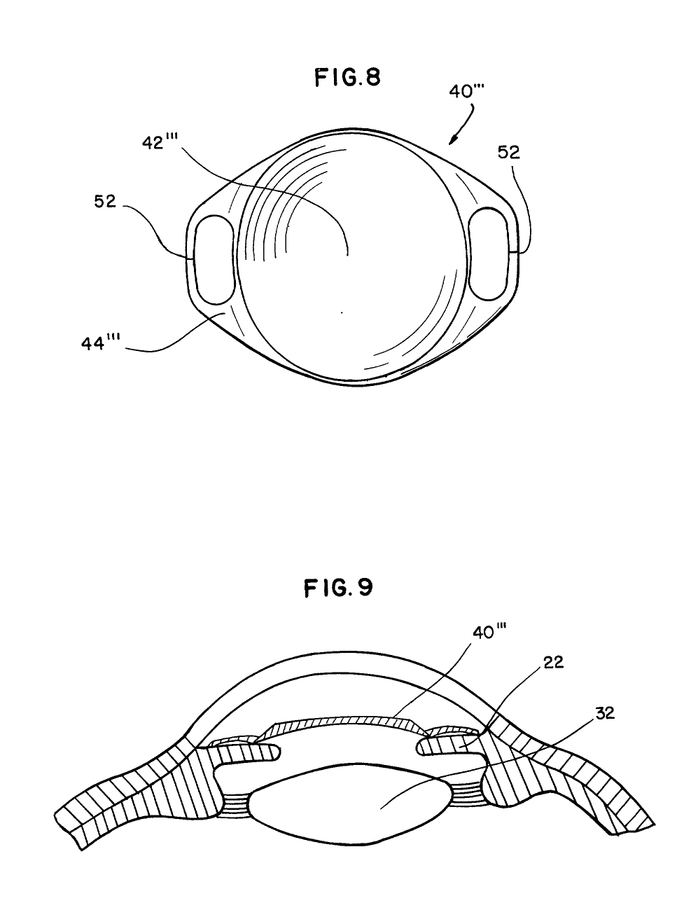

Referring now to FIGS. 8 and 9, a fourth embodiment of the invention is shown in which lens 40″′ is designed for fixation to iris 22. Lens 40′″ can be constructed of a material that is deformable as previously described.

Optic element 42″′ of lens 40″′ can be constructed to be either a negative refracting lens or a positive refracting lens. Additionally, the optic element 42″′ can include a toric element (not shown) for astigmatism correction.

Optic element 42″′ can have a diameter determined by the needs of the wearer, and can range from about 5 mm to about 8 mm, but is preferably from 5.5 to 7.0 mm. The maximum diagonal haptic dimension for lens 40″′ for fixation to iris 22 is also determined by the needs of the wearer, and may range about 7 mm to about 10 mm, but preferably is from 7.5 to 9.0 mm. The vault height for lens 40′″ is in a range around 1 mm, preferably from 0.90 mm to 1.05 mm. Lens 40″′ is fixed to iris 22 by enclavation of midperipheral iris stroma in gap 52 at the distal end of each haptic element 44′″.

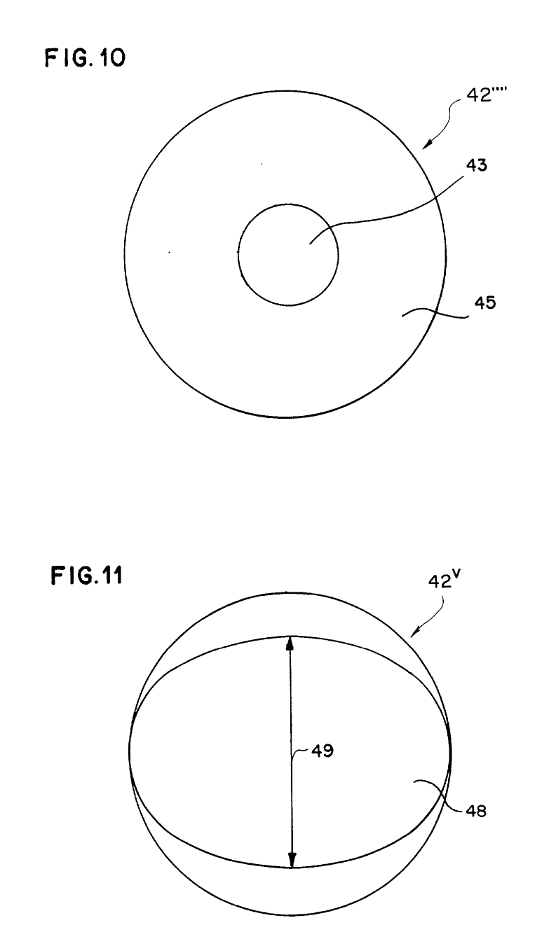

FIG. 10 illustrates an optic element 42″″ of a lens for treatment of presbyopia. Optic element 42″″ for the treatment of presbyopia can be used in conjunction with any of the lens embodiments previously described and placed in any chamber of the eye as previously described. Optic element 42″″ can be attached to a haptic element (not shown) as previously described in other embodiments of the invention. Optic element 42″″ is noticeably distinguished from an optic element for treatment of other disorders in that there are two different focus zones 43 and 45 of optic element 42″″. Inner focus zone 43 is for distance focusing, and is typically around 2 mm in diameter. Outer focus zone 45 is designed for near focusing, and comprises the remainder of optic element 42″″. Additionally, zone 45 of the optic element 42″″ can be designed to include one or more intermediate and/or distance focus zones (not shown) within the near focusing area.

Finally, referring to FIG. 11, an optic element 42^{v} is shown for the correction of astigmatism. Optic element 42^{v} can be used in conjunction with any of the previously described lenses and placed in any chamber of the eye as previously described. Optic element 42^{v} can be attached to a haptic element (not shown) as previously described in other embodiments of the invention. Toric element 48 of optic element 42^{v} is a cylindrical lens with its steepest radius of curvature in its shortest dimension 49. Toric element 48 is on the anterior surface of optic element 42^{v}. Optic element 42^{v} can be oriented in the eye such that the flattest radius of curvature of toric element 48 aligned with the axis of the plus cylinder of the patient's refraction.

Thus, there has been described an IOL that can treat presbyopia, alone or in combination with myopia, hyperopia, and astigmatism. The disclosed IOL can also correct astigmatism, alone, or in combination with myopia or hyperopia.

A method for preventing or delaying cataract formation by using an IOL that has UV absorbing properties has also been disclosed, as has a method for delaying or preventing the onset of presbyopia by using an IOL with UV absorbing materials. Finally, a method for preventing or delaying the degradation of the IOL has been disclosed by using UV absorbing compounds in the IOL.

Whereas the present invention has been described with respect to specific embodiments thereof, it will be understood that various changes and modifications will be suggested to one of ordinary skill in the art and it is intended that the invention encompass such changes and modifications as fall within the scope of the appended claims.

What is claimed is:

1. An artificial, intraocular lens, comprising:
an optic element made of a deformable material; and
a means for securing said optic element in an eye;
wherein said optic element is UV-light absorbing; and
wherein said optic element is adapted to treat presbyopia.

2. The lens of claim 1 wherein said optic element is negative refracting.

3. The lens of claim 1 wherein said optic element is positive refracting.

4. The lens of claim 1 wherein said optic element includes a toric element adapted to treat astigmatism.

5. The lens of claim 1 wherein said optic element is held in place by contact with a ciliary sulcus of the eye.

6. The lens of claim 1 wherein said optic element is held in place by the interaction between said optic element and a pupil of the eye, caused by a radius of curvature of said lens.

7. The lens of claim 1 having an overall diameter of about 7.5 mm to about 9.0 mm.

8. The lens of claim 1 having an overall diameter of 7.5 mm to 9.0 mm.

9. The lens of claim 1 having a vault height of about 0.9 mm to about 1.05 mm.

10. The lens of claim 1 having a vault height of 0.9 mm to 1.05 mm.

11. The lens of claim 1 wherein said optic element is made of silicone.

12. The lens of claim 1 wherein said optic element is made of hydrogel.

13. The lens of claim 1 wherein said optic element is made of a collagen/acrylic blend.

14. The lens of claim 1 wherein said optic element is made of acrylic.

15. The lens of claim 1 wherein said optic element is made of a collagen/hema (collamer) blend.

16. The lens of claim 1 wherein said optic element absorbs light throughout the range of 300 nm to 400 nm.

17. The lens of claim 1 wherein said optic element is adapted to treat myopia.

18. The lens of claim 1 wherein said optic element is adapted to treat hyperopia.

19. The lens of claim 1 wherein said optic element is adapted to treat astigmatism.

20. The lens of claim 1 wherein said optic element is adapted to treat astigmatism and myopia.

21. The lens of claim 1 wherein said optic element is adapted to treat astigmatism and hyperopia.

22. The lens of claim 1 wherein said securing means comprises a haptic element.

23. The lens of claim 22 wherein said optic element has a diameter of 5.5 mm to 7.0 mm.

24. The lens of claim 22 wherein said haptic element consists of at least one haptic body.

25. The lens of claim 24 wherein said haptic body has a maximum diagonal haptic dimension from 10.0 mm to 15.0 mm.

26. The lens of claim 24 wherein said haptic body has a maximum diagonal haptic dimension from 10.0 mm to 11.8 mm.

27. The lens of claim 24 wherein said haptic body has a maximum diagonal haptic dimension from 10.5 mm to 14.0 mm.

28. The lens of claim 24 wherein said haptic body has a maximum diagonal haptic dimension from 11.5 mm to 15.0 mm.

29. The lens of claim 24 wherein said haptic body includes at least two footplates.

30. The lens of claim 24 wherein said haptic body is made of a flexible material.

31. The lens of claim 24 wherein said haptic body is made of a deformable material.

32. The lens of claim 24 wherein said haptic body is made of a flexible acrylic.

33. The lens of claim 24 wherein said haptic body is made of a flexible PMMA.

34. The lens of claim 24 wherein said haptic body is made of silicone.

35. The lens of claim 24 wherein said haptic body is made of hydrogel.

36. The lens of claim 24 wherein said haptic body is made of a collagen/acrylic blend.

37. The lens of claim 24 wherein said haptic body is made of acrylic.

38. The lens of claim 24 wherein said haptic body is made of collagen/hema (collamer) blend.

39 The lens of claim 24 wherein said haptic body suspends the lens in an anterior chamber of the eye at a vault angle of 2 degrees to 5 degrees.

40. An artificial, intraocular lens, comprising:
an optic element made of a deformable material; and
means for securing said optic element in an eye;
wherein said optic element absorbs UV light; and
wherein said optic element is adapted to treat myopia.

41. The lens of claim 40, wherein said optic element is adapted to treat myopia in combination with astigmatism.

42. An artificial, intraocular lens, comprising:
an optic element made of a deformable material; and
means for securing said optic element in an eye;
wherein said optic element absorbs UV light; and
wherein said optic element is adapted to treat hyperopia.

43. The lens of claim 42, wherein said optic element is adapted to treat hyperopia in combination with astigmatism.

44. A method for delaying the onset of cataract formation, comprising the steps of:
providing a UV-absorbing intraocular lens; and
implanting the intraocular lens in a patient's phakic eye.
